(19) United States
(12) Patent Application Publication
(10) Pub. No.: US 2006/0269483 A1
(43) Pub. Date: Nov. 30, 2006

(54) SEM cathodoluminescent imaging using up-converting nanophosphors

(75) Inventors: Robert Austin, Princeton, NJ (US); Shuang Fang Lim, Singapore (SG); Robert Riehm, Freital (DE)

Correspondence Address: SYNNESTVEDT LECHNER & WOODBRIDGE LLP, P O BOX 592, PRINCETON, NJ, 08542-0592, US

(73) Assignee: The Trustees of Princeton University, Princeton, NJ (US)

(21) Appl. No.: 11/494,157

(22) Filed: Jul. 27, 2006

Related U.S. Application Data
(63) Continuation of application No. PCT/US06/07095, filed on Feb. 28, 2006.
(60) Provisional application No. 60/656,995, filed on Feb. 28, 2005.

(51) Int. Cl. A61K 49/00

(52) U.S. Cl. 424/9.6; 977/927

(57) ABSTRACT

Methods for high resolution tissue imaging in which a tissue to be imaged is labeled with UCP's coupled to probes that bind specifically to biological markers on the tissue; the UCP's are then excited with electrons so that the UCP's emit cathodoluminescent photons; after which the photon emission is converted to a visible image. Methods for measuring water content, blood content or blood oxygenation in tumor tissue are also disclosed.

### CROSS-REFERENCE TO RELATED APPLICATION

The present invention claims priority benefit under 35 U.S.C. §119(e) of U.S. Provisional Application No. 60/656,995 filed Feb. 28, 2005. The present application also claims priority benefit under 35 U.S.C. §120 of International Application No. PCT/US06/07095 filed Feb. 28, 2006. The disclosures of both applications are incorporated herein by reference.

### BACKGROUND OF THE INVENTION

The present invention relates to high resolution tissue imaging. More particularly, the present invention relates to high resolution tissue imaging with up-conversion nanophosphors.

Perhaps the greatest contribution of physics to biology has been the development of techniques that provide imaging of biomolecules and structures at the angstrom to nanometer length scale. X-ray diffraction is the best known of these techniques which also include scanning and electron microscopy and atomic force microscopy. However, these techniques do not have the ability to see within a complex biological structure.

Confocal imaging and two-photon imaging have been developed to see within a biological structure. Some material and molecules can emit light at shorter wavelengths than the exciting photons via a non-linear two-photon process. Two-photon imaging, done using very high peak power at femtosecond duration laser excitation in the infrared (ir) has the distinct advantages of (1) infrared excitation for deep tissue penetration (2) minimal background and (3) spatial resolution attributable to the dependence of the emission to the square of the ir intensity. While two-photon organic dye molecules have proven to be powerful tools in imaging technologies in biology the megawatt peak powers necessary for efficient two-photon excitation has required very expensive femtosecond lasers.

Up-converting phosphors (UCP's) are ceramic materials in which rare earth atoms are embedded in a crystalline matrix. The materials absorb infrared radiation, and up-convert to emit in the visible spectrum with high efficiency. These materials are not true two-photon non-linear materials because the ir photon transition is to a real state involving a rare earth ion and a second ir photon is sequentially absorbed to lift the system to the visible emitting state through energy transfer to a second rare earth ion. The up-conversion mechanism can either be described as sequential excitation of the same atom, or excitation of two centers and subsequent energy transfer.

The emission of UCP's consists of sharp lines characteristic of atomic transitions in a well-ordered matrix. Using different rare earth dopants, a large number of distinctive emission spectra can be obtained. The UCP's high ir-visible conversion cross-section makes them virtually background-free markers.

Fluorescent markers are commonly used for imaging biological samples, which lack intrinsic contrast mechanisms for optical microscopy. Traditional organic dyes and fluorescent proteins have been used successfully for in-vivo imaging, but suffer from a high bleaching rate when used in high intensity cell imaging studies. Incorporating fluorescent dyes into nanoparticles can reduce the bleaching problem. Unfortunately their broad emission bands limit the number of colors that can be clearly discriminated within a single experiment during multi-color imaging. These shortcomings have been overcome by the use of quantum dots. However, quantum dots have toxic components and thus poor biocompatibility.

An advantage of UCP's for biological imaging is that they are not likely to be toxic, unlike selenium-containing quantum dots. The LD₅₀ for rare earth oxides is on the order of 1000 mg/kg while the LD₅₀ values for many selenium oxides are on the order of 1 mg/kg.

UCP's have gained acceptance as reporters in in-vitro biological assays. Zarling, et al., U.S. Pat. No. 5,698,397 discloses UCP's in combination with a probe component that binds preferentially to a biological target to be assayed in-vitro. The disclosure of Zarling et al., U.S. Pat. No. 5,698,397 is incorporated herein by reference. However, tissue imaging with UCP's has been limited by image resolution, which is inherently limited to the resolution of objects no smaller than one-half of the excitation wavelength. This has limited in-vivo imaging, as well as ex-vivo imaging with UCP's of tissue biopsy samples.

Unless image resolution can be improved the use of UCP's in in-vivo will remain impractical and UCP's will only have utility in in-vitro biological assays.

### SUMMARY OF THE INVENTION

The need for higher resolution imaging with UCP's has been met by the present invention. The present invention incorporates the phenomenon of cathodoluminescence of rare earth doped UCP's. Electron bombardment of UCP's produces a cathodoluminescent emission similar to the luminescent emission produced by infrared excitation. Using electron beams instead of photons to excite the UCP's produces image resolution on the order of 2 to 5 nanometers (nm), enabled by the electron optics in a Scanning Electron Microscope (SEM), depending upon the energy of the electron beam.

Consequently, SEM can be used without significant modification to produce images of tissues labeled with UCP's. The tissues can be labeled by conventional techniques with UCP's in combination with a probe component that binds preferentially to biological markers on the tissue to be imaged, such as the UCP—probe combinations disclosed by U.S. Pat. No. 5,698,397. The visible light emission can be observed via conventional light microscopy or an image can be generated using conventional imaging hardware and software.

Therefore, according to one aspect of the present invention, a method is provided for high resolution tissue imaging by labeling a tissue to be imaged with UCP's coupled to probes that bind specifically to biological markers on the tissue; exciting the UCP's with electrons so that the UCP's emit photons in the visible spectrum; and converting the photon emission to a visible image. Nanometer (nm) scale UCP's are preferred, with UCP's having a particle size less than 50 nm capable of penetrating the blood-tissue barrier being more preferred.

Depending upon location, the tissue can be imaged in-vivo via minimally invasive internal instrumentation, or by exposing the tissue to be imaged in a sterile environment to permit the image to be captured. The present invention can further be used to obtain high resolution images of ex-vivo tissue sections of biopsy samples. In addition, one of ordinary skill in the art will understand how the present invention can be applied to the analyte detection techniques of U.S. Pat. No. 5,698,397.

According to one embodiment of this aspect of the present invention, an inexpensive CW diode laser is used to do two-photon based imaging of biologically targeted UCP nanospheres to achieve 3-D image resolution at 200 nm length scale by conventional means, after which the imaged tissue is sectioned and subjected to SEM scanning to produce images with resolution on the order of 2 to 5 nm.

The present invention is thus particularly useful for tumor detection and imaging, wherein the UCP's serve as contrast agents for imaging tumors in human tissue. However, the UCP's can also serve as diagnostic agents as well. The rich spectral emission of UCP's provide diagnostic agent utility, permitting the metabolic state of tumors to be characterized without using multiple and expensive lasers. Because a UCP emits a discrete set of lines, this spectrum emission density can be analyzed using conventional techniques to determine water content, blood content (via hemoglobin (Hb) detection) and Hb oxygenation simultaneously with a single excitation wavelength. Spectra can be produced by a single UCP compound or plurality of compounds excited by either infrared or electron beam excitation of the tumor tissue, or both.

Therefore, according to another aspect of the present invention a method is provided for measuring two or more of water content, blood content or blood oxygenation in tumor tissue by labeling a tissue to be imaged with UCP's coupled to probes that bind specifically to biological markers on a tumor; exciting the UCP's with infrared photons or electrons so that the UCP's emit photons in the visible spectrum; and converting the photon emission to information on two or more of water content, blood content or blood oxygenation via spectral analysis. The analysis can be preformed as the tumor is being imaged using dispersed light emitted from excited UCP's. The spectrum can be produced by either or both infrared and electron beam excitation if the embodiment employing both imaging techniques is being used.

The foregoing and other objects, features and advantages of the present invention are more readily apparent from the detailed description of the preferred embodiments set forth below, taken in conjunction with the accompanying drawings.

### BRIEF DESCRIPTION OF THE DRAWINGS

FIG. 1A depicts a two-photon infrared up-conversion microscopy system;

FIG. 1B depicts an SEM cathodoluminescence microscopy system according to one embodiment of the present invention;

FIG. 2 depicts the cathodoluminescence spectrum of green Y₂O₃: Yb, Er nanoparticles according to the present invention obtained at 30 keV acceleration;

FIG. 3 depicts the power-law dependence of phosphor luminescence on ir intensity for the nanoparticles of FIG. 2; and

FIGS. 4A and 4B depict SEM images according to the present Invention of phosphor fed worms at (A) 336 and (B) 671 times magnification at 20 kV acceleration voltage

### DETAILED DESCRIPTION OF PREFERRED EMBODIMENTS

The subject invention encompasses cathodoluminescent labels that are excited by electrons and subsequently emit electromagnetic radiation at visible frequencies.

In accordance with the present invention, cathodoluminescent up-converting inorganic phosphors are provided for tissue imaging and tumor detection. The up-converting phosphors of the invention may be attached to one or more probe(s) that bind specifically to biological markers in tissues to serve as a reporter (i.e., a detectable marker) of the location of the probe(s). The up-converting phosphors can be attached to various probes, such as antibodies, streptavidin, protein A, polypeptide ligands of cellular receptors, polynucleotide probes, drugs, antigens, toxins, and others. Attachment of the up-converting label to the probe can be accomplished using various linkage chemistries, depending upon the nature of the specific probe.

For example but not limitation, nanocrystalline up-converting lanthanide phosphor particles may be coated with a polycarboxylic acid (e.g., Addition XW 330, Hoechst, Frankfurt, Germany) and various proteins (e.g., immunoglobulin, streptavidin or protein A) can be physically adsorbed to the surface of the phosphor particle (Beverloo et al. (1991) op.cit., which is incorporated herein by reference). Alternatively, various inorganic phosphor coating techniques can be employed including, but not limited to: spray drying, plasma deposition, and derivatization with functional groups (e.g., -COOH, -NH₂-CONH₂) attached by a silane coupling agent to -SiOH moieties coated on the phosphor particle or incorporated into a vitroceramic phosphor particle comprising silicon oxide(s) and up-converting phosphor compositions.

Vitroceramic phosphor particles can be aminated with, for example, aminopropyl-triethoxysilane for the purpose of attaching amino groups to the vitroceramic surface on linker molecules, however other omega-functionalized silanes can be substituted to attach alternative functional groups. Probes, such as proteins or polynucleotides may then be directly attached to the vitroceramic phosphor by covalent linkage, for example through siloxane bonds or through carbon-carbon bonds to linker molecules (e.g., organofunctional silylating agents) that are covalently bonded to or adsorbed to the surface of a phosphor particle. Covalent conjugation between the up-converting inorganic phosphor particles and proteins (e.g., avidin, immunoglobulin) can be accomplished with homobifunctional, or preferably heterobifunctional, crosslinkers.

For example, surface silanization of the phosphors with tri(ethoxy)thiopropyl silane leaves a phosphor surface with a thiol functionality to which a protein (e.g., antibody) or any compound containing a primary amine can be grafted using conventional N-succinimidyl(4-iodoacetyl)aminobenzoate (SIAB) chemistry (Weltman et al. (1983). Other silanization and cross-linking methods compatible with the inorganic phosphors may be used at the discretion of the practitioner.

Nanocrystalline up-converting phosphor particles suitable for use with the present are typically smaller than about 100 nm in diameter, preferably less than about 50 nm in diameter, and more preferably are 5 to 30 nm or less in diameter. It is generally most preferred that the phosphor particles are as small as possible while retaining sufficient quantum conversion efficiency to produce a detectable signal; however, for any particular application, the size of the phosphor particle(s) to be used should be selected at the discretion of the practitioner.

For instance, some applications (e.g., detection of a non-abundant cell surface antigen) may require a highly sensitive phosphor label that need not be small but must have high conversion efficiency and/or absorption cross-section, while other applications (e.g., detection of an abundant nuclear antigen in a permeabilized cell) may require a very small phosphor particle that can readily diffuse and penetrate sub-cellular structures, but which need not have high conversion efficiency. Thus, the optimal size of inorganic phosphor particle is application dependent and selected by the practitioner on the basis of quantum efficiency data for various phosphors of the invention. Such conversion efficiency data may be obtained from available sources (e.g., handbooks and published references) or may be obtained by generating a standardization curve measuring quantum conversion efficiency as a function of particle size.

Up-conversion has been found to occur in certain materials containing rare-earth ions in certain crystal materials. For example, ytterbium and erbium act as an activator couple in a phosphor host material such as barium-yttrium-fluoride. The ytterbium ions act as absorber, and transfer energy non-radiatively to excite the erbium ions. The emission is thus characteristic of the erbium ion's energy levels.

The invention can be practiced with essentially any state-of-the-art up-converting inorganic phosphor. One embodiment employs one or more phosphors derived from one of several different phosphor host materials, each doped with at least rare earth element or activator couple thereof. Suitable phosphor host materials include: sodium yttrium fluoride (NaYF₄), lanthanum fluoride (LaF₃), lanthanum oxysulfide, yttrium oxysulfide, yttrium fluoride (YF₃), yttrium gallate, yttrium aluminum garnet, gadolinium fluoride (GdF₃), barium yttrium fluoride (BaYF₅, BaY₂F₈), and gadolinium oxysulfide. Suitable activator couples are selected from: ytterbium/erbium, ytterbium/thulium, and ytterbium/holmium. Other activator couples suitable for up-conversion may be used. By combination of these host materials with the activator couples, at least three phosphors with at least three different emission spectra (red, green, and blue visible light) are provided. Generally, the absorber is ytterbium and the emitting center can be selected from: erbium, holmium, terbium, and thulium; however, other up-converting phosphors of the invention may contain other absorbers and/or emitters.

Examples of other suitable phosphor particles are described by Riman et al., U.S. Pat. No. 6,699,406, Kane, U.S. Pat. No. 5,891,361 and Ohwaki et al., U.S. Pat. No. 5,541,012. The disclosures of all three patents are incorporated herein by reference.

The molar ratio of absorber to emitting center is at least about 1:1, more usually at least about 3:1 to 5:1, preferably at least about 8:1 to 10:1, more preferably at least about 11:1 to 20:1, and typically less than about 250:1, usually less than about 100:1, and more usually less than about 50:1 to 25:1. Various ratios may be selected by the practitioner on the basis of desired characteristics (e.g. chemical properties, manufacturing efficiency, quantum efficiency, absorption cross-section, excitation and emission wavelengths, or other considerations). The ratio(s) chosen will generally also depend upon the selected absorber-emitter couple(s) and can be calculated from reference values in accordance with desired characteristics.

For absorber-emitter couples, the optimum ratio of absorber (e.g., ytterbium) to the emitting center (e.g., erbium, thulium, or holmium) varies, depending upon the specific absorber/emitter couple. For example, the absorber to emitter ratio for Yb:Er couples is typically in the range of about 20:1 to about 100:1, whereas the absorber to emitter ratio for Yb:Tm and Yb:Ho couples is typically in the range of about 500:1 to about 2000:1. These different ratios are attributable to the different matching energy levels of Er, Tm, or Ho with respect to the Yb level in the crystal. For most applications, up-converting phosphors may conveniently comprise about 10-30% Yb and either about 1-2% Er, about 0.1-0.05% Ho, or about 0.1-0.05% Tm, although other formulations may be employed.

Inorganic phosphors of the invention typically have emission maxima that are in the visible range. For example, specific activator couples have characteristic emission spectra: ytterbium-erbium couples have emission maxima in the red or green portions of the visible spectrum, depending upon the phosphor host; ytterbium-holmium couples generally emit maximally in the green portion, ytterbium-thulium typically have an emission maximum in the blue range, and ytterbium-terbium usually emit maximally in the green range. For example, Y_{.80}Yb_{.19}Er_{.01}F₂ emits maximally in the green portion of the spectrum.

Although up-converting inorganic phosphor crystals of various formulae are suitable for use in the invention, the following formulae, provided for example and not to limit the invention, are generally suitable:
Na(YₓYb_{y}Er_{z})F₄: x is 0.7 to 0.9, y is 0.09 to 0.29, and z is 0.05 to 0.01;
Na(YₓYb_{y}Ho_{z})F₄: x is 0.7 to 0.9, y is 0.0995 to 0.2995, and z is 0.0005 to 0.001; and
Na(YₓYb_{y}Tm_{z})F₄: x is 0.7 to 0.9, y is 0.0995 to 0.2995, and z is 0.0005 to 0.001.
(YₓYb_{y}Er_{z})O₂S: x is 0.7 to 0.9, y is 0.05 to 0.12; z is 0.05 to 0.12.
(Y_{.86}Yb_{.08}Er_{.06})₂O₃ is a relatively efficient up-converting phosphor material.

For example, various phosphor material compositions capable of up-conversion are suitable for use in the invention are shown in Table I.

TABLE 1
Phosphor Material Compositions

| Host Material Absorber Ion | Emitter | Ion | Color |
|---|---|---|---|
| Oxysulfides (O₂S) | | | |
| Y₂O₂S | Ytterbium | Erbium | Green |
| Gd₂O₂S | Ytterbium | Erbium | Red |
| La₂O₂S | Ytterbium | Holmium | Green |
| Oxyhalides (OX_{y}) | | | |
| YOF | Ytterbium | Thulium | Blue |
| Y₃OCl₇ | Ytterbium | Terbium | Green |
| Fluorides (Fₓ) | | | |
| YF₃ | Ytterbium | Erbium | Red |
| GdF₃ | Ytterbium | Erbium | Green |
| LaF₃ | Ytterbium | Holmium | Green |
| NaYF₃ | Ytterbium | Thulium | Blue |
| BaYF₅ | Ytterbium | Thulium | Blue |
| BaY₂F₈ | Ytterbium | Terbium | Green |
| Gallates (GaₓO_{y}) | | | |
| YGaO₃ | Ytterbium | Erbium | Red |
| Y₃Ga₅O₁₂ | Ytterbium | Erbium | Green |
| Silicates (SiₓO_{y}) | | | |
| YSi₂O₅ | Ytterbium | Holmium | Green |
| YSi₃O₇ | Ytterbium | Thulium | Blue |

In addition to the materials shown in Table I and variations thereof, aluminates, phosphates, and vanadates can be suitable phosphor host materials. In general, when silicates are used as a host material, the conversion efficiency is relatively low. In certain uses, hybrid up-converting phosphor crystals may be made (e.g., combining one or more host material and/or one or more absorber ion and/or one or more emitter ion).

Inorganic phosphor particles can be milled to a desired average particle size and distribution by conventional milling methods known in the art. However, milling crystalline materials has several weaknesses. With milling, the particle morphology is not uniform, as milled particles result from random fracture of larger crystalline particles. Because the sensitivity of a detection assay using up-converting inorganic phosphors depends on the ability to distinguish between bound and unbound phosphor particles, it is preferable that the particles be of identical size and morphology.

The size, weight, and morphology of up-converting nanocrystalline phosphor particles can affect the number of potential binding sites per particle and thus the potential strength of particle binding to reporter and/or analyte. Monodisperse submicron spherical particles of uniform size can be generated by homogeneous precipitation reactions at high dilutions. For example, small yttrium hydroxy carbonate particles are formed by the hydrolysis of urea in a dilute yttrium solution. Similarly, up-converting inorganic phosphors can be prepared by homogeneous precipitation reactions in dilute conditions. For example, (Y_{.86}Yb_{.08}Er₀₆)₂O₃ was prepared as monodisperse spherical particles in the submicron size range by precipitation. Other methods for the preparation of nanoparticles are disclosed in U.S. Pat. No. 6,699,406.

However, after precipitation it is typically necessary to anneal the oxide in air at about 1500 C., which can cause faceting of the spherical particles and generate aggregate formation. Faceting can be substantially reduced by converting the small spherical particles of the oxide or hydroxy carbonate precursor to the oxysulfide phase by including a polysulfide flux for annealing. Using this technique, highly efficient oxysulfide particles in the 300 to 400 nm diameter range were prepared as a dispersion in water. Sonication can be used to produce a monodisperse mixture of discrete spherical particles. After fractionation and coating, these particles can be used as up-converting reporters. This general preparative procedure is suitable for preparing much smaller phosphor particles (e.g., 100 nm diameter or smaller).

Frequently, such as with phosphors having an oxysulfide host material, the phosphor particles are preferably dispersed in a polar solvent, such as acetone or DMSO and the like, to generate a substantially monodisperse emulsion (e.g., for a stock solution). Aliquots of the monodisperse stock solution may be further diluted into an aqueous solution (e.g., a solution of avidin in buffered water or buffered saline).

It was found that washing phosphors in acetone or DMSO improved suspendability of inorganic phosphor particles in water. In particular, the phosphor particles prepared with polysulfide flux are preferably resuspended and washed in hot DMSO and heated for about an hour in a steam bath then allowed to cool to room temperature under continuous agitation. The phosphor particles may be pre-washed with acetone (typically heated to boiling) prior to placing the particles in the DMSO. Hot DMSO-treated phosphors were found to be reasonably hydrophilic and form stable suspensions.

A Microfluidizer™ (Microfluidics Corp.) can be used to further improve the dispersion of particles in the mixture. DMSO-phosphor suspen-sions can be easily mixed with water, preferably with small amounts of surfactant present. In general, polysaccharides (e.g., guar gum, xanthan gum, gum arabic, alginate, guaiac gum) can be used to promote particle deaggregation. In a variation, particles are washed in hot DMSO and serially diluted into 0.1% aqueous gum arabic solution, and appears to virtually eliminate water dispersion problems of phosphors. Re-suspended phosphors in organic solvent, such as DMSO, are typically allowed to settle for a suitable period (e.g., about 1-3 days), and the supernatant which is typically turbid is used for subsequent conjugation.

Ludox™ is a colloidal silica dispersion in water with a small amount of organic material (e.g., formaldehyde, glycols) and a small amount of alkali metal. Ludox™ and its equivalents can be used to coat up-converting phosphor particles which can subsequently be fired to form a ceramic silica coating which cannot be removed from the phosphor particles, but which can be readily silanized with organofunctional silanes (containing thiol, primary amine, and carboxylic acid functionalities) using standard silanization chemistries (Arkles, B., Silicon Compounds: Register and Review, (5th Edition, Anderson, R. G., Larson, G. L., and Smith, C., eds., Huls America, Piscataway, N.J., 1991), 59-64.

UCP particles can be coated or treated with surface-active agents (e.g., anionic surfactants such as Aerosol OT). For example, particles may be coated with a polycarboxylic acid (e.g., Additon XW 330, Hoechst, Frankfurt, Germany or Tamol, see Beverloo et al. (1992) op.cit.) to produce a stable aqueous suspension of phosphor particles, typically at about pH 6-8. The pH of an aqueous solution of phosphor particles can be adjusted by addition of a suitable buffer and titration with acid or base to the desired pH range. Depending upon the nature of the coating, some minor loss in conversion efficiency of the phosphor may occur as a result of coating, however the power available in an electron beam excitation source can compensate for any reduction in conversion efficiency and ensure adequate phosphor emission.

In general, preparation of inorganic phosphor particles and linkage to binding reagents is performed essentially as described in Beverloo et al. (1992) op.cit., and Tanke U.S. Pat. No. 5,043,265. Alternatively, a water-insoluble polyfunctional polymer which exhibits glass and melt transition temperatures well above room temperature can be used to coat the up-converting phosphors in a nonaqueous medium. For example, such polymer functionalities include: carboxylic acids (e.g., 5% acrylic acid/95% methyl acrylate copolymer), amine (e.g., 5% aminoethyl acrylate/95% methyl acrylate copolymer) reducible sulfonates (e.g., 5% sulfonated polystyrene), and aldehydes (e.g., polysaccharide copolymers).

The phosphor particles are coated with water-insoluble polyfunctional polymers by coacervative encapsulation in non-aqueous media, washed, and transferred to a suitable aqueous buffer solution to conduct the heterobifunctional crosslinking to a protein (e.g., antibody) or polynucleotide probe molecule. An advantage of using water-insoluble polymers is that the polymer microcapsule will not migrate from the surface of the phosphor upon aging the encapsulated phosphors in an aqueous solution (i.e., improved reagent stability). Another advantage in using copolymers in which the encapsulating polymer is only partially functionalized is that one can control the degree of functionalization, and thus the number of biological probe molecules which can be attached to a phosphor particle, on average. Since the solubility and coacervative encapsulation process will depend on the dominant nonfunctionalized component of the copolymer, the functionalized copolymer ratio can be varied over a wide range to generate a range of potential crosslinking sites per phosphor, without having to substantially change the encapsulation process.

A preferred functionalization method employs heterobifunctional crosslinkers that can be made to link the biological macromolecule probe to the insoluble phosphor particle in three steps: (1) bind the crosslinker to the polymer coating on the phosphor, (2) separate the unbound crosslinker from the coated phosphors, and (3) bind the biological macromolecule to the washed, linked polymer-coated phosphor. This method prevents undesirable crosslinking interactions between biological macromolecules and so reduces irreversible aggregation as described by Tanke et al. Examples of suitable heterobifunctional crosslinkers, polymer coating functionalities, and linkable biological macromolecules include, but are not limited to:

| Coating | Heterobifunctional | Biological |
|---|---|---|
| Functionality | Crosslinker | Macromolecule |
| carboxylate | N-hydroxysuccimide 1-ethyl-3- | Proteins (e.g., Ab, |
| | (3-dimethyl-aminopropyl) | avidin) |
| | carbodiimide (EDC) | |
| primary amine | N-5-azido-2-nitrobenzoyl | All having 1° amine |
| | oxysuccimide (ANB-NOS) | |
| | N-succinimidyl (4-iodoacetyl) | |
| | aminobenzoate (SIAB) | |
| thiol (reduced | N-succinimidyl (4-iodoacetyl) | Proteins |
| sulfonate) | aminobenzoate (SIAB) | |

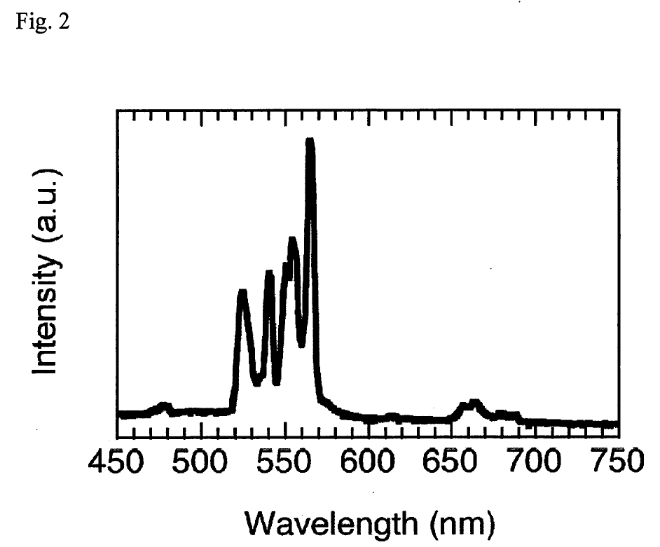

Detection and quantitation of inorganic up-converting phosphor(s) is generally accomplished by: (1) illuminating a sample suspected of containing up-converting phosphors with an electron beam, and (2) detecting catyhodoluminescent radiation at one or more emission wavelength band(s). The cathodoluminescence spectrum of green Y₂O₃: Yb, Er nanoparticles obtained at 30 keV acceleration is depicted in FIG. 2.

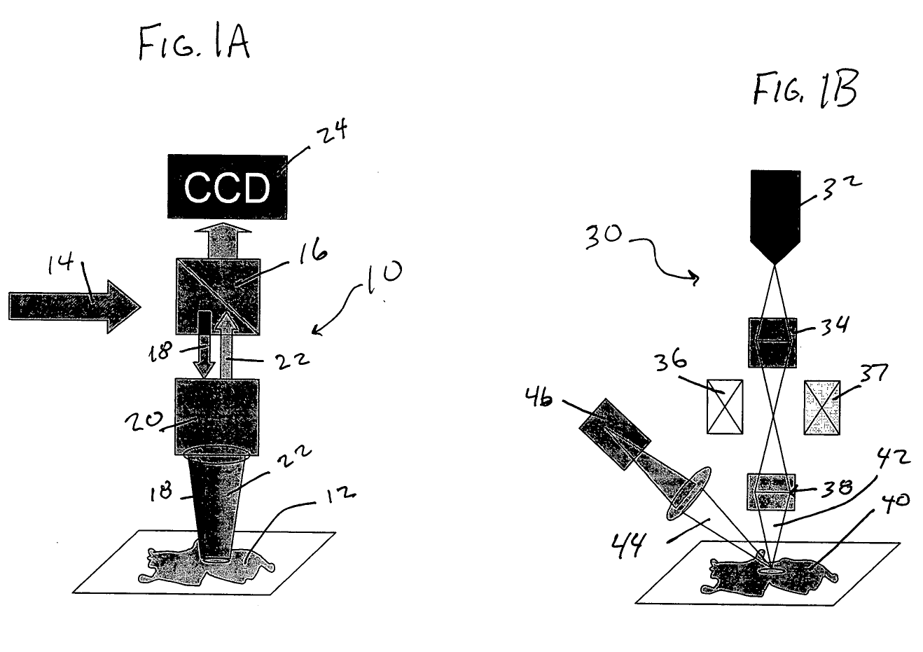

Illumination of the sample is produced by exposing the sample to an electron beam, such as the 20-30 keV beam produced by a Scanning Electron Microscope (SEM). One example of a suitable SEM is a Philips XL30 (FEI, Hillsboro, Oreg.). An SEM cathodoluminescence microscopy system is depicted in FIG. 1B. SEM 30 consists of electron gun 32, condenser lens system 34, scan coils 36 and 37 and objective lens 38. Tissue specimen 40 containing UCP's (not shown) is raster scanned by electron beam 42. The UCP's emit visible light 44, the photons of which are detected by photomultiplier tube 46, from which the total photon counts for each beam position are measured to convert the optical signal into an electronic signal.

Once the optical signal from the sample is converted into an electronic one, a standard, composite video signal can be developed by conventional means and displayed as an image on a television monitor (not shown). The image can be manipulated and enhanced through standard image processing software.

Detection and quantitation of luminescence from excited UCP's can be accomplished by a variety of means in addition to photomultiplier devices. Various means of detecting emission(s) can be employed, including but not limited to: avalanche photodiodea, charge-coupled devices (CCD), CID devices, photographic film emulsions, photochemical reactions yielding detectable products, and visual observation (e.g., fluorescent light microscopy). Detection can employ time-gated and/or frequency-gated light collection for rejection of residual background noise.

Time-gated detection is generally desirable, as it provides a method for recording long-lived emission(s) after termination of illumination; thus, signal(s) attributable to phosphorescence or delayed fluorescence of an up-converting phosphor is recorded, while short-lived autofluoresence and scattered illumination light, if any, is rejected. Time-gated detection can be produced either by specified periodic mechanical blocking by a rotating blade (i.e., mechanical chopper) or through electronic means wherein prompt signals (i.e., occurring within about 0.1 to 0.3 microseconds of termination of illumination) are rejected (e.g., an electronic-controlled, solid-state optical shutter such as Pockel's or Kerr cells).

Up-converting phosphors typically have emission lifetimes of approximately a few milliseconds (perhaps as much as 10 ms, but typically on the order of 1 ms), whereas background noise usually decays within about 100 ns. Therefore, when using a pulsed excitation source, it is generally desirable to use time-gated detection to reject prompt signals. Because up-converting phosphors are not subject to photobleaching, very weak emitted phosphor signals can be collected and integrated over very long detection times (continuous illumination or multiple pulsed illumination) to increase sensitivity of detection.

A two-photon infrared up-conversion microscopy system 10 is depicted in FIG. 1A. The up-converting phosphors (not shown) in tissue specimen 12 are excited with an externally mounted CW IR diode laser (not shown). IR beam 14 is routed through the microscope's dichroic beam splitter 16. IR beam 18 passes through objective lens 20 onto the tissue specimen, exciting the UCP's. The UCP's emit visible light beam 22, which is transmitted back to the dichroic beam splitter, which images the visible light on a CCD 24.

The electronic signal is likewise developed into a standard, composite video signal that can be developed by conventional means and displayed as an image on a television monitor (not shown). The image can also be manipulated and enhanced through standard image processing software, but with a resolution on the order of 200 nm, as opposed to the 2 to 5 nm resolution obtained through cathodoluminescent imaging.

It is possible, however, to reconstruct a 3 dimensional view of sample 12 with the two-photon infrared up-conversion microscopy system. The reconstruction is formed by stepping through sample 12 at small intervals, making an image of the sample at each interval. The multiple sequential images are transferred to an external graphics machine (not shown) for reconstruction of the sample in 3 dimensions. These 3-D images can then be rotated to give different perspectives of the data sets, leading to a better understanding of the samples on a larger scale before the samples are sectioned and imaged using with higher resolution using the SEM microscopy system depicted in FIG. 1B.

Thus, the ability to use electron beam excitation for stimulating UCP's provides several advantages. First, a 100-fold improvement in image resolution is obtained, so objects as small as 2 to 5 nm can be imaged. Second, the inventive method can be implemented using conventional SEM equipment, optical imaging hardware and software.

When the tissue to be imaged is a tumor, the up-converting phosphors of the invention are attached to one or more probe(s) that bind specifically to tumors tissue. The UCP's serve as contrast agents for tumor detection. The UCP's can also be employed as tumor diagnostic agents by analysis of a portion of the visible light emitted by the tissue sample during SEM cathodoluminescence microscopy or during two-photon infrared up-conversion microscopy. UCP spectral emissions permit the metabolic state of tumors to be analyzed using conventional techniques to determine water content, blood content (via hemoglobin (Hb) detection) and Hb oxygenation simultaneously.

Spectra can be produced by a single UCP compound or plurality of compounds excited by either infrared or electron beam excitation of the tumor tissue, or both. The spectral analysis can be preformed as the tumor is being imaged using the dispersed light emitted from excited UCP's. The spectrum can be produced by either or both infrared and electron beam excitation if the embodiment employing both imaging techniques is being used.

Imaging compositions may be prepared in which the up-converting phosphors of the invention with one or more probe(s) attached that bind specifically to biological markers in tissues are suspended in a tissue-compatible carrier. The composition may be administered systemically or locally to a patient for tissue-imaging purposes by means of a syringe or catheter. Other imaging or contrast agents may also be present. The tissue may be imaged in situ or a biopsy may be performed for external analysis. The composition may also be applied ex-vivo to a biopsy sample for imaging purposes.

When the tissue is tumor tissue, the composition may also be used to identify tissue to be removed during cancer surgery and confirm that the tumor was completely removed. That is, any tumor tissue remaining will have UCP's present from the composition that was first administered to image the tumor. The surgical site can be illuminated with infrared light and any tumor tissue remaining will emit visible light from the UCP's present.

Although the present invention has been described in some detail by way of illustration for purposes of clarity of understanding, it will be apparent that certain changes and modifications may be practiced within the scope of the claims. The broad scope of this invention is best understood with reference to the following examples, which are not intended to limit the invention in any manner.

### EXAMPLE

The viability of the UCP nanoparticles for biological imaging was confirmed by imaging the digestive system of the nematode worm C. elegans. C. elegans was chosen because of the size amenable to optical microscopy. The short life cycle and rapid growth enables quick chartering of genetic mutations.

The phosphors were prepared by homogeneous precipitation. An aqueous solution of Y(NO₃)₃·6H₂O (50 mM), Yb(NO₃)₃·5H₂O (1 mM), Er(NO₃)₃·5H₂O (0.5 mM), and urea (15mM) (all Sigma-Aldrich, St. Louis, Mo.) was heated to boiling with vigorous agitation, which led to thermal hydrolysis. The premixing of the reactants prior to hydrolysis reduced the possibility of any concentration gradient, ensuring that precipitates formed had a narrow size distribution. The reaction was stopped by lowering the temperature of the solution in an ice bath. The size of the precipitates was controlled by the concentration of the salts and the time of the reaction. The resulting precipitate was then washed six times with de-ionized water, followed by centrifugation after every wash. The product was dried at 150° C. for two hours and the crystalline oxide was obtained by annealing at 1000° C. for 2 hours. UCP's synthesized under these conditions exhibit green upconversion. A similar synthesis with a different relative rare earth concentration yields red upconversion.

We imaged nanoparticles in a scanning electron microscope (Philips XL30, FEI, Hillsboro, Oreg.) with a 10 kV electron beam after coating the particles with a 5 nm gold film. Energy-Dispersive X-ray Spectrometry (EDX) was conducted on a PGT-IMIX PTS EDX system in order to perform elemental analysis as well as mapping.

N2 wild type C. elegans were grown on Nematode Growth Medium (NGM) agar plates at 25° C., which had been seeded with E. coli strain OP50 that had been cultured in 1.05 L broth. The OP50 strain was cultured in L broth at 37° C. overnight. A phosphor dispersion consisting of 0.5 mg phosphor, with a mean particle size of 150 nm, was prepared in 1.0 ml NGM buffer (3 g NaCl, 1 ml 1 M CaCl₂, 1 ml 1 M MgSO₄, 25 ml 1 M KPO₄ buffer, 975 ml DI water, Sigma Aldrich). The phosphors were dispersed by sonication and pippetted onto a C. elegans dish that was 72 hours old, allowing for three hours uptake.

For ir imaging purposes, suitable worms were transferred into an eppendorf tube containing NGM buffer and concentrated by short centrifugation. They were then pipetted onto an agar bed that was afterwards sandwiched between two cover slips. A sufficient amount of sodium azide was added in order to immobilize the worms. For SEM imaging, 100 microliters of Poly-L-lysine solution (0.1 w/V in water and 0.01 Thimerosal, Sigma Aldrich) was applied onto a precleaned glass slide and air dried over 30 hours. Subsequently, another 50 microliters of Poly-L-lysine was applied over the previously dried layer, followed immediately by transferring of the C. elegans from agar plates under sterile conditions onto the liquid Poly-L-lysine layer and allowed to air dry over 24 hours. A final 50 microliter aliquot of Poly-L-lysine was applied onto the C. elegans/Poly-L-lysine and air dried.

Dehydration was performed through a series of ethanol/water mixtures, beginning with 25%, 50% and 100% ethanol (anhydrous, 200 proof, 99.5%, Sigma Aldrich). About 50 microliters of ethanol/water mixture was applied each time, followed by air drying before the next application. The glass slides were cleaved into 1 cm squares and mounted onto aluminium stubs with the use of carbon tape. Graphite adhesive was also applied to the edges of the substrates in order to enhance charge dissipation. The mounted substrates were then coated with 4 nm thick Iridium in order to prevent charging during imaging.

Imaging of the C. elegans by up-conversion phosphorescence with IR excitation was performed using an inverted microscope with a 20x, 0.4 N.A. microscope objective (Nikon, Melville, N.Y.), coupled to an intensified CCD camera (Princeton Instruments, Trenton, N.J.). The worms were imaged in both bright-field and epi-fluorescence geometries. The latter was enabled by a custom-made fluorescence filter set (Chroma technology, Rockingham, Vt.), and a 20-W infrared LED laser array. The illumination intensity was about 10 W/mm². The dependence of the luminescence intensity was determined by integrating the emission from one particle in the field of view, and varying the illumination intensity. Up-conversion luminescence spectra were collected using a fiber-coupled CCD spectrometer Ocean Optics, Dunedin, Fla.

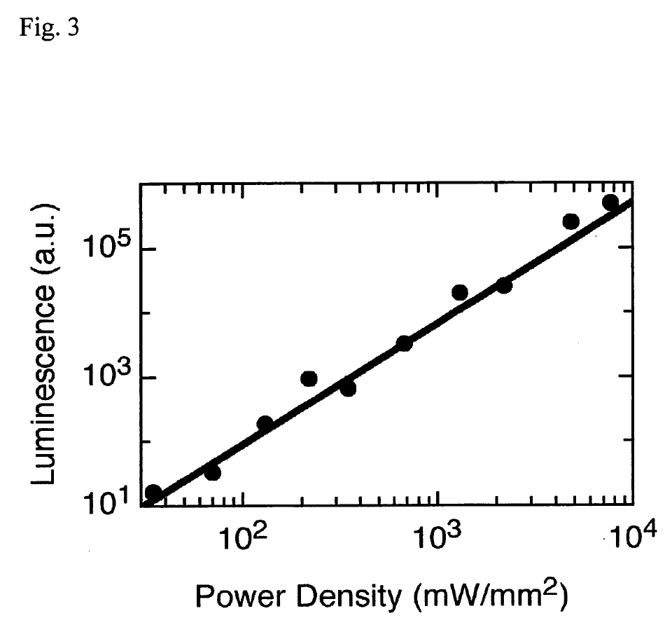

The dependence of the fluorescence intensity on the illumination power is plotted in FIG. 3. We find a power-law dependence of the luminescence on the ir-illumination intensity, with an exponent of 1.88. The imaging of C. elegans was performed at the high-power end of the presented curve.

The cathodoluminescent (CL) properties of the UCP's was investigated in a Scanning Electron Microscope (SEM). The CL spectrum measured at 30 keV electron acceleration of the green phosphors is shown in FIG. 1. It is observed from the figure that emission occurs virtually from the same energy levels as during photoluminescent emission, except for differences with regards to relative intensities among the transition lines.

We successfully inoculated UCP nanoparticles into C. elegans by placing them on an agar plate that has been wetted with a 150 nm sized particle suspension in Nematode Growth Medium (NGM) buffer. We were able to see individual, point-like UPC particles, and found that the imaging resolution was limited by the combination of the microscope objective and the camera.

The phosphors were easily visible in the intestines, with most particles found beyond the pharynx, extending to the rectum. When food is made available to the phosphor fed worms, the phosphors are secreted in under two hours. Thereafter, these worms continue feeding and appear unaffected by the prior ingestion of the phosphors. Hence, it has been demonstrated that UCP's are biocompatible and non-toxic, which make them ideal candidates as bio-labels.

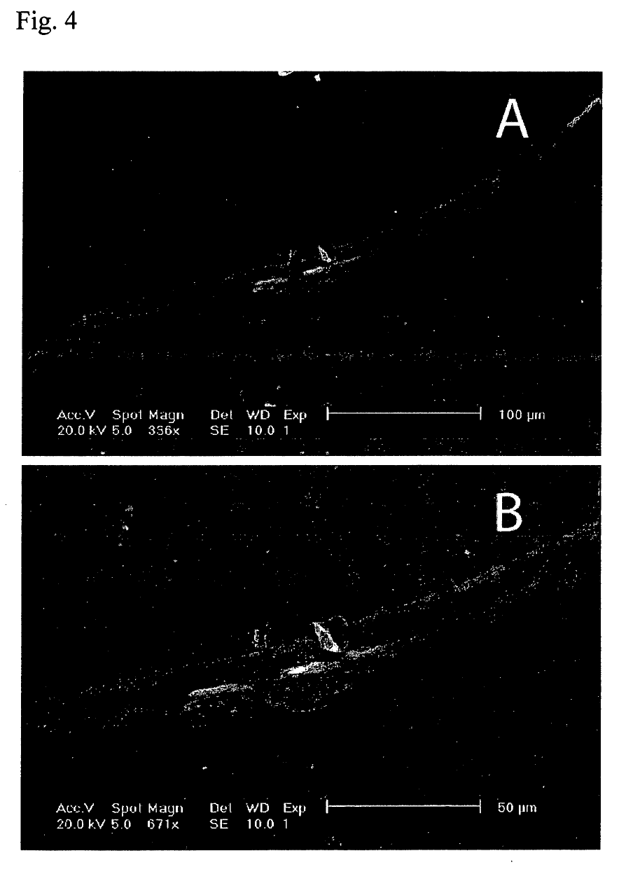

For SEM microscopy, the worms were mounted onto cleaned and pretreated glass slides which ensures sticking of the worm. Systematic dehydration was carried out in a series of ethanol:water mixtures. A 4 nm thick Iridium metal coating was sputtered onto the prepared worms prior to SEM imaging. FIGS. 4a and 4B show SEM images of a phosphor fed worm at different magnifications. The phosphors typically glow intensely and stably within the worm in both the secondary and backscattered (not shown) imaging mode. The phosphors are observed to glow brightly inside the worm in the SEM image at 20 kV acceleration voltage.

We have shown that UCP's can be excited by electron impact. This opens up new possibilities of using higher resolution imaging techniques such as SEM with UCP's used as bio-labels.

While a number of preferred embodiments of the invention and variations thereof have been described in detail, other modifications and methods of use will be readily apparent to those of skill in the art. Accordingly, it should be understood that various applications, modifications and substitutions may be made of equivalents without departing from the spirit of the invention or the scope of the claims.

What is claimed is:

1. A method for high resolution tissue imaging comprising labeling a tissue to be imaged with UCP's coupled to probes that bind specifically to biological markers on said tissue; exciting said UCP's with electrons so that said UCP's emit cathodoluminescent photons; and converting the photon emission to a visible image.

2. The method of claim 1, wherein said UCP's have a particle size less than about 50 nm.

3. The method of claim 2, wherein said UCP's have a particle size between about 5 and about 30 nm.

4. The method of claim 1, wherein said probe is selected from the group consisting of antibodies, streptavidin, protein A, polypeptide ligands of cellular receptors, polynucleotide probes, drugs, antigens and toxins.

5. The method of claim 1, wherein said UCP's comprise a phosphor host material selected from the group consisting of sodium yttrium fluoride, lanthanum fluoride, lanthanum oxysulfide, yttrium oxysulfide, yttrium fluoride, yttrium gallate, yttrium aluminum garnet, gadolinium fluoride, barium yttrium fluoride, and gadolinium oxysulfide.

6. The method of claim 1, wherein said UCP's comprise an activator couple selected from the group consisting of ytterbium/erbium, ytterbium/thulium and ytterbium/holmium.

7. The method of claim 1, wherein said UCP's comprise an activator couple, wherein the absorber is ytterbium and the emitting center is selected from the group consisting of erbium, holmium, terbium and thulium.

8. The method of claim 7, wherein said emitting center is erbium.

9. The method of claim 1, wherein said electrons have an energy between about 20 and about 30 keV.

10. The method of claim 1, wherein said electrons are produced by a Scanning Electron Microscope.

11. The method of claim 1, wherein the wavelength of said photons is in the visible spectrum.

12. The method of claim 1, wherein the photon emission is converted to a visible image using a photomultiplier tube.

13. A method for measuring two or more of water content, blood content or blood oxygenation in tumor tissue comprising, labeling tumor tissue with UCP's coupled to probes that bind specifically to biological markers on said tumor; exciting said UCP's with infrared photons or electrons so that said UCP's emit luminescent or cathodoluminescent photons; and converting the photon emission to information on two or more of water content, blood content or blood oxygenation via spectral analysis.

14. The method of claim 13, wherein said analysis is preformed as the tumor is being imaged using dispersed light emitted from excited UCP's.

15. The method of claim 13, wherein said UCP's are excited with infrared photons.

16. The method of claim 13, wherein said UCP's are excited with electrons.

17. The method of claim 16, wherein said electrons have an energy between about 20 and about 30 keV.

18. The method of claim 13, wherein said electrons are supplied by a Scanning Electron Microscope.

19. The method of claim 13, wherein water content, blood content or blood oxygenation in tumor tissue are all measured by spectral analysis.

20. The method of claim 13, wherein said UCP's have a particle size less than about 50 nm.

21. The method of claim 13, wherein said UCP's comprise an activator couple, wherein the absorber is ytterbium and the emitting center is selected from the group consisting of erbium, holmium, terbium and thulium.
